(19) United States
(12) Patent Application Publication
(10) Pub. No.: US 2005/0282777 A1
(43) Pub. Date: Dec. 22, 2005

(54) Dextrinized, Saccharide-Derivatized Oligosaccharides

(75) Inventors: Richard L. Antrim, Solon, IA (US); Frank W. Barresi, Coralville, IA (US); Roger McPherson, Muscatine, IA (US); Jiao Wang, Muscatine, IA (US)

Correspondence Address: BANNER & WITCOFF, LTD., TEN SOUTH WACKER DRIVE, SUITE 3000, CHICAGO, IL, 60606, US

(73) Assignee: Grain Processing Corporation, Muscatine, IA (US)

(21) Appl. No.: 11/184,989

(22) Filed: Jul. 20, 2005

Related U.S. Application Data
(62) Division of application No. 10/601,912, filed on Jun. 23, 2003.
(60) Provisional application No. 60/390,570, filed on Jun. 21, 2002.

(51) Int. Cl. A61K 31/721; C08B 37/02

(52) U.S. Cl. 514/59; 536/112

(57) ABSTRACT

Disclosed are saccharide-derivatized oligosaccharides. The derivatized oligosaccharides preferably are prepared by extruding a malto-oligosaccharide mixture with a saccharide or mixture of saccharides having a DP ranging from 1 to 4. The products are low in digestibility, and thus in various embodiments are suitable for use as bulking agents, for controlled energy release products, and for other purposes.

### RELATED APPLICATION

This application claims priority to prior provisional application Ser. No. 60/390,570, filed Jun. 21, 2002.

### TECHNICAL FIELD

The invention is in the field of starch and starch derivatives, and in particular, is in the field of oligosaccharides. More particularly, the invention is directed towards an oligosaccharide compound and composition that are useful as low-calorie bulking agents and slow energy release products.

### BACKGROUND

Many substances are used in the manufacture of foods intended for persons and animals who must restrict their intake of carbohydrates or calories. Such substances generally should be of low caloric value and of a generally non-nutritive nature. In addition, such substances must not be toxic or unwholesome. The foods or animal feeds produced using such substances preferably are formulated such that they resemble higher calorie products in texture, taste and physical appearance.

Among such substances are synthetic sweeteners. When a synthetic sweetener such as saccharin or aspartame is used in a dietetic food as a substitute for sugar, the other physical properties which would have been imparted by sugar, such as appearance, bulk mass, and texture, must also be imparted to the dietetic food by a separate ingredient. For instance, saccharin and aspartame both are substantially sweeter than sugar. It is often necessary to provide a low-calorie, non-nutritive carrier so that the bulk mass, appearance, and texture of the added sweetener approximates that of sugar.

The prior art has provided numerous such bulking agents. One such bulking agent that is well known in literature is polydextrose, as is taught, for instance, in U.S. Pat. Nos. 3,766,165 and 3,876,794 (both to Rennhard). Polydextrose is a product of melt polymerization of glucose or maltose, generally using edible acids, such as citric acid, as catalysts and cross-linking agents. Polydextrose has a substantially reduced caloric value relative to sugar (about 1 Kcal/gm), or about 25% that of dextrose. As such, polydextrose may be used as a bulking agent in connection with synthetic sweeteners and other applications.

Although polydextrose is satisfactory for many purposes as a non-nutritive bulking agent, there exist several practical difficulties concerning the use of this material. For instance, the production of polydextrose is not without difficulty. Polydextrose generally is prepared in a condensation reaction that is performed under harsh conditions. As such, the condensation reaction often results in a dark-colored product that has an undesirable acidic and bitter flavor. Numerous efforts have been made to address this problem. For instance, efforts to improve on the manufacturing process of polydextrose have been provided. As taught, for instance, in EP 404,227 (to Coöperatieve Vereniging Suiker Unie V.A.) and in U.S. Pat. No. 5,015,500 (to Elmore), various extrusion techniques for polydextrose have been taught. Another reference, U.S. Pat. No. 5,558,899 (to Kuzee et al.), purports to disclose the production of polydextrose via use of microwave energy. Other references purport to disclose methods to improve the taste or flavor of polydextrose. For instance, U.S. Pat. No. 4,622,233 (to Torres) purportedly teaches peroxide bleaching of polydextrose in an alcohol solvent. U.S. Pat. No. 4,948,596 (to Bunich et al.) purportedly discloses a liquid/liquid extraction process for purifying polydextrose. U.S. Pat. No. 4,956,458 (to Luo et al.) is said to disclose another process said to be useful for purifying polydextrose. U.S. Pat. Nos. 5,091,015 (to Bunich); 5,677,593 (to Guzek et al.); and 5,831,082 (to An et al.) purport to teach chromatographic methods for purifying polydextrose. U.S. Pat. No. 5,573,794 (to DuFlot) purports to disclose glucose oxidase treatment of polydextrose followed by ion exchange chromatography. Finally, U.S. Pat. Nos. 5,601,863 (to Borden et al.) and 5,424,418 (to Duflot et al.) disclose hydrogenated polydextrose.

All of the foregoing approaches to polydextrose production are somewhat limited in utility. One principal drawback common to all of these approaches is that the polydextrose produced by any process typically includes substantial quantities of undesired color and flavor components, and substantial effort is required to reduce the levels of such components to acceptable levels. Moreover, the polydextrose product that is obtained in a typical condensation reaction has a low molecular weight. It would be desirable to have a low calorie bulking agent that has the properties of a higher molecular weight product such as a maltodextrin. More recently, to address this latter concern, a number of patents, including U.S. Pat. Nos. 5,264,568 (to Yamada et al.); 5,358,729; 5,364,652; and 5,430,141 (all to Ohkuma et al.); and EP 368,451 (to Matsutani Chemical Industries Co. Ltd.) purport to disclose a product, commonly known as FIBERSOL, that is formed by starch pyrodextrinization followed by enzymatic hydrolysis to leave an undigestive carbohydrate remnant. It is said that the disclosed product can be hydrogenated and/or ion exchanged to give a final product with reduced calorie content and soluble fiber benefits. This product is higher is molecular weight than most polydextroses, and therefore has properties that rival maltodextrins. However, the product also suffers from low processing yields, significant processing complexities, and high final cost.

In addition to such low- or non-caloric products, there is a demand for a carbohydrate product that can be digested slowly. Ideally, the carbohydrate product should be fully digestible, yet should deliver calories evenly for an extended period of time. Typically, carbohydrates that are fully digestible are digested rapidly, causing a spike in blood glucose levels soon after ingestion (a hyperglycemic state) followed by a drop in blood glucose level (a hypoglycemic state) due to overexpression of insulin. For some people, potential ill effects such as increase risk of cardiovascular disease and hypoglycemic related side effects such as blurred vision, loss of consciousness, and diminished mental acuity can result from such fluctuation in blood glucose levels.

The prior art has provided numerous controlled energy released products. Hydrogenated starch hydrolysates such as LYCASIN® (Roquette Freres) and HYSTAR® (SPI Polyols) are examples of such products. It is known that these products are digested more slowly then their non-hydrogenated counterparts, because the digestion products of a hydrogenated starch hydrolyzate are glucose and sorbitol, and the sorbitol component of the mixture is digested more slowly than glucose. See Dwivedi, Food Science & Technology Books, Vol. 17 pp. 165-183 (1986). One drawback of hydrogenated starch hydrolysates is that they have relatively high osmblality and are associated with high level of sorbitol and maltitol digestion products that can cause cramping and diarrhea.

Another document, International Publication WO 96/31129, discloses a mixture of rapidly digestible, slowly digestible, and non-digestible products. For instance, this document teaches that a combination of rapidly digested carbohydrate with a slowly digested complex carbohydrate such as raw cornstarch in conjunction with proteins and fats can be used in the control of blood glucose levels. The slowly digested product is a raw starch, which is not fully digested and, because of its lack of cold water solubility, is only amenable to use in solid products.

Chemically modified starches, such as oxidized, dextrinized, and etherified starches also have been examined as candidates for control energy release (see, e.g., J. Agric. Food Chem. 47:4178 (1999)). In general, it has been found that the more chemically modified a material is, the less digestible the material is. Most of these products tend to have no digestibility or very low digestibility, and thus may be considered to be resistant starches or soluble fiber.

It is a general object of the present invention to provide an oligosaccharide product. In some preferred embodiments, it is an object to provide a low-calorie oligosaccharide product that does not suffer from the same disadvantages as polydextrose and that can be produced more readily and inexpensively than the enzymatically treated starch pyrodextrinization product hereinbefore described. In other embodiments, it is a general object to provide an oligosaccharides product that releases nutritional energy slowly in comparison to glucose.

### THE INVENTION

It has now been found that dextrinized saccharide-derivatized oligosaccharides may be prepared, and that such products can function as bulking agents or as slow energy release compounds. Preferably, an oligosaccharide, which most preferably is a malto-oligosaccharide, is dextrinized by extrusion in the presence of a saccharide. The dextrinized oligosaccharide product and the process for its preparation offer a number of completely unexpected properties and advantages not heretofore realized. For instance, in some embodiments the product has low digestibility, and thus is suitable in a number of applications as a bulking agent, a product carrier, or the like. In other embodiments, the product can be made to release nutritional energy slowly relative to glucose. The product does not require large amounts of acid for catalysis, and in some instances, the product may be prepared with no acid catalysis whatsoever. Not as much polymerization is required for production of the product as is required in the preparation of polydextrose, and thus the harsh reaction conditions typically required for polydextrose production are not required. The preferred process for production of the derivatized product is simple, with a high tolerance for moisture content in the starting materials. Thus, there is no need to take expensive steps to avoid moisture uptake in the starting materials. The product has a higher molecular weight than most commercially available polydextrose products, thus making the product similar in properties to many maltodextrins and therefore suitable for use in more applications than is polydextrose. Finally, and perhaps most surprisingly, color components and undesired flavor components formed in the process are kept to a minimum, and these undesired components readily can be removed.

In accordance with one embodiment of the invention, a dextrinized oligosaccharide is provided. The oligosaccharide comprises the product of dextrinization of a saccharide having a degree of polymerization (DP) of at least 5. Preferably, the oligosaccharide is extruded in the presence of a saccharide having a DP of 1 to 4. The lower molecular weight saccharide will function as a lubricant. Moreover, it has been found that the dextrinized oligosaccharides will be derivatized to an extent with the lower molecular weight saccharide. In preferred embodiments, the invention provides a saccharide-derivatized oligosaccharide mixture which comprises the reaction product of a low-DP saccharide product with a mixture of malto-oligosaccharides in which at least a portion of the malto-oligosaccharides in the mixture have a degree of polymerization greater than 5. Also provided by the invention are a process for preparing a saccharide product and a process for preparing a mixture of oligosaccharides as set forth hereinbelow.

In accordance with another embodiment, a starch or limit dextrin is derivatized with a saccharide, preferable a saccharide having a degree of polymerization ranging from 1 to 4. Any suitable starch or limit dextrin can be used in conjunction with the invention.

### DETAILED DESCRIPTION OF THE PREFERRED EMBODIMENTS

Generally, in preferred embodiments, the invention contemplates the dextrinization of an oligosaccharide. The oligosaccharide preferably is a malto-oligosaccharide. By “malto-oligosaccharide” is contemplated any species comprising two or more saccharide units linked predominantly via 1-4 linkages, and including maltodextrins and syrup solids. Maltodextrins have a dextrose equivalent value (DE) of less than 20 or whereas syrup solids have a DE of 20 or greater. In preferred embodiments, at least 50% of the saccharide units in the malto-oligosaccharide are linked via 1-4 linkages. More preferably, at least about 60% of the saccharide units are linked via 1-4 linkages; and even more preferably, at least about 80% of the saccharide units are so linked. Malto-oligosaccharides may include saccharide species having an odd or even DP value, and may include some dextrose (DP 1). The invention is applicable to derivatization of malto-oligosaccharide species in which at least a portion of the malto-oligosaccharides in the mixture have a DP value greater than 5. Preferably, at least one of the malto-oligosaccharides species in the mixture has a DP value of 8 or more. Most preferably, at least one species has a DP value of least 10. In preferred embodiments in the invention, at least 70% of the malto-oligosaccharide species in the mixtures have a degree of polymerization greater than 5; even more preferably, at least about 80% of the malto-oligosaccharides species in the mixture have a degree of polymerization greater than 5.

Suitable malto-oligosaccharides are sold as maltodextrins under the trademark MALTRIN® by Grain Processing Corporation of Muscatine, Iowa. The MALTRIN® malto-oligosaccharides are malto-oligosaccharide products, each product having a known typical DP profile. Suitable MALTRIN® maltodextrins may serve as starting materials in accordance with the present invention and include MALTRIN® M040, MALTRIN® M050, MALTRIN® M100, MALTRIN® M150, and MALTRIN® M180. Typical DP profiles of the subject MALTRIN® maltodextrins are set forth in the following table:

| DP | | | | | |
|---|---|---|---|---|---|
| pro- | Typical DP profile (% dry solids basis) | | | | |
| file | M180 | M150 | M100 | M050 | M040 |
| DP > | 46.6 ± 4% | 54.7 ± 4% | 67.8 ± 4% | 90.6 ± 4% | 88.5 ± |
| 8 | | | | | 4% |
| DP 8 | 3.9 ± 2% | 4.8 ± 1.5% | 4.5 ± 1.5% | 1.5 ± 1% | 2.0 ± |
| | | | | | 1% |
| DP 7 | 9.5 ± 2% | 9.1 ± 1.5% | 7.0 ± 1.5% | 1.5 ± 1% | 2.4 ± |
| | | | | | 1% |
| DP 6 | 11.4 ± 2% | 8.4 ± 1.5% | 6.1 ± 1.5% | 1.4 ± 1% | 1.8 ± |
| | | | | | 1% |
| DP 5 | 5.9 ± 2% | 4.7 ± 1.5% | 3.3 ± 1.5% | 1.3 ± 1% | 1.3 ± |
| | | | | | 1% |
| DP 4 | 6.4 ± 2% | 5.5 ± 1.5% | 3.7 ± 1.5% | 1.1 ± 1% | 1.4 ± |
| | | | | | 1% |
| DP 3 | 8.3 ± 2% | 6.7 ± 1.5% | 4.2 ± 1.5% | 1.0 ± 1% | 1.4 ± |
| | | | | | 1% |
| DP 2 | 6.2 ± 2% | 4.8 ± 1% | 2.5 ± 1% | 0.8* ± 1% | 0.9* ± |
| | | | | | 1% |
| DP 1 | 1.8 ± 1.5% | 1.3 ± 1% | 0.7* ± 1% | 0.8* ± 1% | 0.3* ± |
| | | | | | 1% |

*MINIMUM VALUE = 0%

Each of these maltodextrins has at least 45% DP 10 or greater malto-oligosaccharide. Other suitable malto-oligosaccharide starting materials can include other malto-oligosaccharides, such as MALTRIN® M440, MALTRIN® M4510, MALTRIN® M580, MALTRIN® M550, and MALTRIN® M700, as well as corn syrup solids, such as MALTRIN® M200, MALTRIN® M250, and MALTRIN® M360. The malto-oligosaccharides can be ion-exchanged or hydrogenated. One method for hydrogenating mixtures of malto-oligosaccharides is disclosed in published PCT Application WO 99/36442 (to Grain Processing Corporation). The malto-oligosaccharide starting materials further may be derivatized, as disclosed, for instance, in U.S. Pat. No. 6,380,379.

The invention is not limited to malto-oligosaccharide species, and indeed, any suitable malto-oligosaccharide may be employed as a starting material in conjunction with the present invention. In another embodiment of the invention, for instance, the starting material is a starch. Any suitable starch may be used in conjunction with the invention. Examples starch include corn, potato, waxy material, tapioca rice, and the like. One suitable cornstarch is sold under the trademark B200 by Grain Processing Corporation of Muscatine, Iowa. In accordance with another embodiment of the invention, the starting material is a limit dextrin. Limit dextrins are discussed in more detail in copending application Ser. No. 09/796,027. The starting material may be another dextrin that comprises a starch that has been partially hydrolyzed by an alpha amylase enzyme but not to the theoretical or actual limit. Such dextrins are referred to herein as “prelimit dextrins.”

The oligosaccharide or other starting material is dextrinized in the presence of a lower molecular weight saccharide, i.e., a saccharide having a degree of polymerization ranging from 1 to 4. Mixtures of malto-oligosaccharides typically include some DP 1-4 saccharides, but in most cases additional saccharide should be added. Preferably, the saccharide is dextrose, optionally in combination with one or more other saccharides, such as maltose, maltotriose or maltotetraose. If a mixture of saccharides is employed, the average DP of the mixture should be in the range of 1 to 4, preferably 1 to 3, and even more preferably 1 to 2. Mixtures of saccharides that can be employed include MALTRIN® M250 and MALTRIN® M360. It is contemplated that these latter products, which include some lower order saccharides and some oligosaccharides having a DP greater than four, may themselves be extruded and thus may be deemed themselves to be a mixture of the saccharide and oligosaccharides. Alternatively, the derivatizing saccharide may be maltose, maltotriose or maltotetraose in the presence or absence of dextrose. However, dextrose is the preferred saccharide. Preferably, if a mixture of saccharides is employed, the saccharide includes dextrose or maltose in an amount of at least 50% by weight of the mixture. It has been found that in the derivatization reaction, the dextrose serves as a processing aid in addition to being a reactant. In some embodiments of the inventions, a hydrogenated starch hydrolyzate, preferably sorbitol, but also possibly maltitol or a higher order hydrogenated starch hydrolyzate, is used in connection with the low-order saccharide. Such a hydrogenated starch hydrolyzate serves as a chain terminator to limit the formation of high molecular weight molecules and also serves as a plasticizer and processing aid in connection with the reaction. When a hydrogenated starch hydrolyzate is used, it preferably is present in an amount ranging from about 50 to about 95% by weight of the added saccharide component.

The reaction preferably is catalyzed using an acid, which is present in an amount ranging from about 0.01 to about 1.5% by weight, preferably about 0.1 to about 0.5% by weight of the total reaction mixture. The preferred acid is citric acid, which should be used in an amount ranging of about 0.125% by weight of the total reaction mixture. Other suitable acids include acetic acid, adipic acid, fumaric acid, gluconic acid, lactic acid, malic acid, phosphoric acid, and tartaric acid.

The oligosaccharides and saccharide preferably are present in a ratio of about 4:1 (oligosaccharide:saccharide). It is contemplated that the 4:1 ratio is approximate, and may be varied depending on the reactants chosen and/or the reaction conditions employed. It has been observed that as the molecular weight of the oligosaccharide increases, the amount of dextrose or other lower saccharide also should increase. More generally, the amount of dextrose or the lower order saccharide should be about 10% to about 30% by weight of the total reaction mixture, with the oligosaccharide constituting essentially the rest of the reaction mixture.

All of the foregoing weight percentages are expressed on a dry solids basis. It has been found that moisture may be present in the reaction mixture without detracting from the derivatization reaction. It is contemplated that moisture may be present in an amount of up to about 50% by weight. Preferably, any moisture is present in a substantially lower amount, such as about 5 to 10% by weight, to permit moisture to be added during extrusion of the mixture of starting materials. In any event, the moisture content of the starting materials is not critical.

The dextrinized oligosaccharides preferably are formed from the foregoing ingredients in the absence of other ingredients. It is contemplated that other derivatizing agents or other catalysts or the like could be employed.

In accordance with the invention, the starting materials, which include the oligosaccharides or other starting material, the saccharide, any hydrogenated starch hydrolyzate, any catalyzing acid, and any other material may be reacted in any suitable fashion to dextrinize the oligosaccharides or other starting material. The dextrinization should be sufficient to convert at least a portion of the highly digestible 1-4 bonds present in the starting material to other bonds. Generally, the application of heat and/or material energy is necessary to dextrinize the starting material. Most preferably, the starting materials are combined and reacted in an extruder. The extruder can include any conveying device in which temperature, vacuum, water, and the starting materials can be introduced with adequate mixing to result in derivatization. For example, a Wenger TX-57 Twin Screw Extruder can be used to generate an acceptable product. The extruder may be operated under any suitable conditions. Generally, extrusion conditions require barrel temperatures that range from about 25° C. to about 220° C., with the maximum barrel temperature more preferably in a range of about 140° to 180° C. The internal sample temperature at the dye head of the extruder can be in a range of 160° C. to 275° C., but preferably remains between the range 190° to 230° C. The revolutions permitted for the extruder can vary between 25 and 500 rpm, with optimal conditions in the 300 to 425 rpm range. Vacuum optionally can be applied to the system; if applied, up to 18 inches of mercury (0.4 atm) can be used. The foregoing set of conditions is by no means meant to be exhaustive or limiting, but to the contrary these conditions are provided for general guidance. The actual extruder conditions can vary widely depending on the starting materials and the type of extruder being used.

The amount of lower saccharide should be selected relative to the amount of oligosaccharide starting material such that the product that is extruded from the extruder barrel appears as a straw-colored, low-density solid that crumbles and dissolves easily. Preferably, the amount of saccharide chosen is sufficient to yield such product without charring, but insufficient to result in a product that is in liquid form. Excess dextrose will result in poor processing conditions. The exact amount of dextrose chosen in a given extrusion reaction is a matter well within the purview of one of ordinary skill in the art. When a mixture of malto-oligosaccharides is reacted with the saccharide, the mixture of malto-oligosaccharides is “derivatized,” by which is contemplated the derivatization of at least a portion of the oligosaccharides having a DP greater than 5 (and possibly the derivatization of lower order saccharides in the malto-oligosaccharide mixture).

The dextrinized, derivatized oligosaccharide product prepared by the foregoing process is easily solubilized and requires little downstream processing to substantially reduce the levels of undesired color and flavor components. For example, the product can be dissolved in water and treated with 0.5 to 10% carbon, such as SA-30 carbon from Westvaco, for up to 4 hours at 75° C. The material then may be filtered and otherwise treated, for instance, by spray-drying. Spray-drying of the decolorized material yields an off-white final product with a bland, malto-oligosaccharide taste. Further processing such as chemical bleaching, ion exchange, membrane filtration, or hydrogenation can also be used to improve the final color of the product. If an ion exchanged or hydrogenated-ion exchanged starting material is used, downstream processing to remove color and flavor components may be facilitated or made altogether not necessary.

The resulting product may have a low caloric value relative to dextrose. It is believed that this is because the product will be unaffected by amylolytic enzymes such as amylo-1-4-glucosidases, amylo-1-4, 1-6-glucosidases, amylo-1-4-dextrosidases, and amylo-1, 4 maltosidases, as well as alpha-beta-glucosidases, sucrase, and phosphorylase. Thus, the product may be substantially inert to digestion by mammalian enzymes, although mammalian intestinal flora may be able to ferment a portion of the product and make fermentation products available for digestion. The product alternatively may be substantially digestible, but digestible slowly relative to glucose. It is believed that relatively low levels of chemical modification of the starting material will produce a product having some non 1-4 linking bonds, (e.g., 1-2, 1-3, or 1-6 bonds) that are resistant to enzymatic degradation in the digestive system. The majority of the bonds will be subject to enzymatic hydrolysis. Because of the random nature of the new bonds that are formed, the overall product will be digested slowly relative to the starting material (and relative to glucose) due to less enzymatic recognition of the hydrolyzable segments of the material.

The product thus prepared is suitable for use in numerous applications. Typical uses are found in low calorie spreadable foods such as jellies, jams, preserves, marmalades, sugar-fruits, compotes, fruit garnish, fillings, and fruit butters; in frozen food compositions, including ice cream, iced milk, sherbet, and water ices; in baked goods, such as cakes, cookies, pastries, and other foodstuff containing wheat or other flour; in icings, candy, and chewing gum; in beverages, such as non-alcoholic soft drinks, root extracts, fruit or vegetable juices, or mineral water; in syrups; in toppings, sauces, and puddings; in salad dressings; and so forth. The invention finds particular use as a bulking agent for dry low calorie sweeteners such as saccharin, sucralose, or aspartame. The product also finds use as a carrier or excipient. More generally, the product may be used as a bulking agent for products such as soaps, cosmetics, food products, animal feeds, and so forth. It is further contemplated that the product may find other uses. For instance, in embodiments of the invention where the product is digestible slowly, the product may be used in sports and nutritional drinks and solid food products such as energy bars. The product may be used in products for individuals with diabetes.

Moreover, the product thus prepared is suitable for use as texturizing agents, thickening and/or gelling agents, emulsifying agents, filling or encapsulating agents, particularly in food products, in pharmaceutical or veterinary products, and in sugar-free confectioneries (e.g., chewy pastes, caramels, toffees, chocolates, fudges and nougats), Which may comprise viscosity-promoting agents (gum arabic, gelatin, modified starches, maltodextrins, carrageenans, agar, pectin, and the like), humectants (sorbitol, glycerin), egg white, and flavorings. Moreover, the product can be used in compositions intended to be ingested by humans and animals, e.g., those administered orally, e.g., soups, fibre-enriched fruit-based compositions, fibre-enriched drinks, e.g., fiber-enriched low-calorie drink (e.g., fibre-enriched soft drinks), mayonnaise, biscuits, lozenges, preparations based on milk, fermented milks, and foodstuff fermentations. The fermented food compositions at which the present invention is directed can be of animal or vegetable origin and can also be intended for animal nutrition, particularly as silage-making compositions. The product can be in the form of dessert creams or yogurts directly consumed by the patient or which can be administered by a tube. Moreover, use in dietetic or hygiene products such as, e.g., elixirs, cough syrups, tablets or pills, hygienic solutions for oral cavity, toothpastes and tooth gels.

Sports drinks, for example, are available in several compositions. In one embodiment, the composition is a ready-to-drink aqueous solution that can be packaged in single serving or larger containers. The components are mixed together in sterile, filtered, or carbonated water and packaged for sale. In another embodiment, the components are mixed in an aqueous solution in a concentrated form. An aliquot of the concentrated solution is then mixed with a pre-measured amount of water to prepare the beverage. In another embodiment, the composition is a dry powder form in which the dried components are mixed together and milled or mixed in aqueous solution and dried by one of the methods described below. An aliquot of the dried components is mixed with a pre-measured amount of water to prepare the beverage. The dry powder may be loose or fashioned into tablets which can be easily added to a pre-measured amount of water to prepare the beverage.

Sports drinks can additionally comprise, other sugars, e.g., trehalose. Other suitable carbohydrates include mono- di- and polysaccharides. Suitable mono-saccharides include, but are not limited to, fructose, mannose, glucose and galactose. Suitable disaccharides include, but are not limited to, sucrose, maltose and lactose. Suitable polysaccharides include, but are not limited to, maltodextrins and those described in European Patent Specification Publication No. 223,540.

Moreover, sports drinks can comprise suitable salts, which include, but are not limited to, sodium, potassium, magnesium and calcium. European Patent Application Publication No. 587,972 provides an extensive discussion of such salts and suitable concentrations thereof. Suitable sources of the salts include, but are not limited to, sodium chloride, potassium phosphate, potassium citrate, magnesium succinate and calcium pantothenate. Salts are optional, and, as discussed above, are primarily beneficial in increasing fluid intake by the intestinal tract. Thus, the amount of salts added is preferably suitable to affect an increase in fluid intake without resulting in an unpalatable drink.

In addition to carbohydrates and salts, the sports drink may contain various other nutrients. These include, but are not limited to, vitamins, minerals, amino acids, peptides and proteins. Suitable vitamins include, but are not limited to, vitamin C, the B vitamins, pantothenic acid, thiamin, niacin, niacinamide, riboflavin, iron and biotin. Minerals include, but are not limited to, chromium, magnesium and zinc. Preferably, amino acids are included rather than peptides and proteins which require digestion prior to absorption. Suitable amino acids include, but are not limited to, the twenty amino acids utilized by humans. U.S. Pat. No. 4,871,550 discusses preferred amino acids. The effective amounts of the various nutrients are known in the art and are not described in detail herein. Other ingredients including, but not limited to, coloring, flavor, artificial sweeteners and preservatives may also be added. Suitable amounts and types of all ingredients described herein are known in, the art and are not described in detail herein. It is within the skill of one in the art to prepare a beverage formulation having suitable concentrations of all the components.

Energy bars can additionally comprise nutrients, such as calcium, vitamin D, Vitamins B12, folic acid, B6, niacin, C or E, iron and zinc. Moreover energy bars can comprise lipoic acid and carnitine, optionally in combination with coenzyme Q10 and/or creatine, in a timed release formulation to provide a steady supply of the nutrients to the mitochondria which work 24 hours a day. Such additional components can be in any suitable form, e.g., coating a core comprising the micronutrient(s) and excipients (coated system) and incorporating the micronutrient(s) into a matrix (matrix system). Coated systems involve the preparation of product-loaded cores and coating the cores with release rate-retarding materials. Product-loaded cores can be formulated as microspheres, granules, pellets or core tablets. There are many known core preparation methods, including, but not limited to, 1) producing granules by top spray fluidized bed granulation, or by solution/suspension/powdering layering by Wurster coating, 2) producing spherical granules or pellets by extrusion-spheronization, rotary processing, and melt pelletization; 3) producing core tablets by compression and coating with a release rate-retarding material; 4) producing microspheres by emulsification and spray-drying.

Matrix systems embed the micronutrient in a slowly disintegrating or non-disintegrating matrix. Rate of release is controlled by the erosion of the matrix and/or by the diffusion of the micronutrient(s) through the matrix. In general, the active product substance, excipients and the release rate-retarding materials are mixed and then processed into matrix pellets or tablets. Matrix pellets can be formed by granulation, spheronization using cellulosic materials, or by melt pelletization using release retardant materials, while matrix tablets are prepared by compression in a tablet press. An example of a cellulosic material is hydroxypropylmethylcellulose as the release rate retarding material.

Coated or matrix pellets can be filled into capsules or compression tabletted. The rate of release can be further modified by blending coated or matrix pellets with different release rates of the same product to obtain the desired product release profile. Pellets containing any of lipoic acid, carnitine, coenzyme Q10 or creatine can be blended to form a combination product.

More generally, the invention is also contemplated to be suitable for use in connection with the uses disclosed in published U.S. patent application Nos. 20030077368 (entitled “Fibre-enriched drinks”; 20030039740 (“Composition for enteral nutrition comprising fibres”); 20020192355 (“Fibre-enriched table sweeteners”); 20020192344 (“Process for preparing a low-calorie food”); 20020182299 (“Process for manufacturing fibre-enriched fruit-based compositions and compositions thus obtained”); and 20020136798 (“Carbon-containing additive for foodstuff fermentations and food compositions containing it”) and in published Australian application no. AU 199963030 A1 (“Branched maltodextrins and method of preparing them”). The materials disclosed in connection with the present application may be substituted for the materials purportedly described in the foregoing publications.

The following examples are provided to illustrate the present invention, but should not be construed as limiting the scope of the invention.

### EXAMPLES 1-18

### Preparation of Dextrinized Saccharide-Derivatized Oligosaccharides

These examples illustrate the preparation of various saccharide-derivatized oligosaccharides. A blend of maltodextrins/anhydrodextrose/citric acid (87.5%/12.5%/1.0%) Was made by mixing 1312.5 grams of MALTRIN® M100 and other MALTRIN® products with 187.5 grams of anhydrodextrose and 15 grams of citric acid. These materials were thoroughly mixed in a Hobart mixer. The resulting blend was then manually fed into an 18 mm twin screw Leistritz extruder. The extruder barrel temperature was monitored in six zones, according to the following table:

| | |
|---|---|
| Zone 1 | 32° C. |
| Zone 2 | 81° C. |
| Zone 3 | 180° C. |
| Zone 4 | 201° C. |
| Zone 5 | 201° C. |
| Zone 6 (die head) | 198° C. |

Low shear extruder screws were used. The extruder screw speed rate was 100 rpm. A single, 3 mm dye opening was used at the die head. The percent motor load for the extruded sample was 55%.

In each instance, a straw-colored solid material was extruded. The material was allowed to cool and ground to a golden yellow powder. Each sample was analyzed for molecular weight, percentage digestibility, and color. Molecular weight calculations were done via HPLC-SEC TRISEC (VISCOTEK® oration, Houston Tex.). For control purposes, dextrose was extruded. The products were prepared according to the following table:

| Percent Composition | | | | | | | |
|---|---|---|---|---|---|---|---|
| | | | Dex- | | | | |
| | | MD | trose | Citric | Max. | Shaft | |
| Ex- | MD | wt. % | wt. % | Acid | Temp. | Speed | % Motor |
| ample | Type | (dsb) | (dsb) | wt. % | (° C.) | (RPM) | Load |
| Control | N/A | 0 | 100 | 1 | 200 | 25 | 39 |
| Control | N/A | 0 | 100 | 1 | 220 | 25 | 16 |
| 2 | | | | | | | |
| 1 | M360 | 50 | 50 | 1 | 200 | 100 | 38 |
| 2 | M250 | 50 | 50 | 1 | 200 | 100 | 38 |
| 3 | M250 | 93.75 | 6.25 | 1 | 200 | 100 | 40 |
| 4 | M250 | 96.875 | 3.125 | 1 | 200 | 100 | 55 |
| 5 | M250 | 100 | 0 | 1 | 200 | 200 | 55 |
| 6 | M200 | 100 | 0 | 1 | 200 | 200 | 75 |
| 7 | M200 | 50 | 50 | 1 | 200 | 100 | 30 |
| 8 | M180 | 50 | 50 | 1 | 200 | 100 | 40 |
| 9 | M150 | 50 | 50 | 1 | 200 | 100 | 55 |
| 10 | M100 | 50 | 50 | 1 | 200 | 100 | 55 |
| 11 | M100 | 75 | 25 | 1 | 200 | 100 | 38 |
| 12 | M100 | 87.5 | 12.5 | 1 | 200 | 100 | 55 |
| 13 | M100 | 93.75 | 6.25 | 1 | 180 | 100 | 75 |
| 14 | M100 | 96.875 | 3.125 | 1 | 200 | 100 | 55-60 |
| 15 | M070 | 50 | 50 | 1 | 200 | 100 | 45 |
| 16 | H- | 50 | 50 | 1 | 180 | 100 | 40 |
| | M180 | | | | | | |
| 17 | M040 | 50 | 50 | 1 | 200 | 100 | 52 |
| 18 | M040 | 87.5 | 12.5 | 1 | 200 | 100 | 50 |

MD = maltodextrin type
DE = dextrose equivalent
H-M180—hydrogenated M180

Percentage maltodextrin and dextrose were expressed on a dry solids basis per total weight (maltodextrin and dextrose). The dextrose value represents dextrose added to the malto-oligosaccharide.

Upon analysis, the following results were obtained:

| | | Molecular Weight | | | |
|---|---|---|---|---|---|
| Example | DE | Mw | Mn | % digest | UV 420 Color |
| Control | 15.4 | 1860 | 1050 | 7.54 | 2.6 |
| Control 2 | 6.8 | 3700 | 790 | 4.58 | 14.2 |
| 1 | 21.7 | 3010 | 900 | 24.77 | 9.1 |
| 2 | 17.3 | 2940 | 1390 | 16.75 | 27 |
| 3 | 11.2 | 5470 | 2150 | 9.24 | 58.5 |
| 4 | 10.6 | 5720 | 870 | 9.42 | 63.3 |
| 5 | 13.8 | 3390 | 1350 | 35.3 | 14.2 |
| 6 | 11.9 | 6500 | 950 | 14.28 | 118 |
| 7 | 14.6 | 4530 | 740 | 18.71 | 19.5 |
| 8 | 18 | 4980 | 1300 | 21.67 | 10.3 |
| 9 | 18.9 | 4510 | 1890 | 22.11 | 6.8 |
| 10 | 13.2 | 5050 | 630 | 14.42 | 13.8 |
| 11 | 14 | 4850 | 1770 | 22.44 | 9.7 |
| 12 | 11.8 | 4700 | 2150 | 16.59 | 14.8 |
| 13 | 10.2 | 5600 | 2250 | 7.23 | 52.8 |
| 14 | 10.6 | 7650 | 2350 | 5.12 | 136 |
| 15 | 12.8 | 5280 | 1610 | 13.83 | 14.6 |
| 16 | 21.8 | 4780 | 460 | 49.42 | 2.03 |
| 17 | 12.9 | 5290 | 1360 | 13.61 | 17.2 |
| 18 | 10.5 | 6440 | 2450 | 6.82 | 96 |

% digest = 3 hour digestibility adapted from J. S. White et al. , J. Food Sci. , Vol. 53, No. 4, 1988, pp. 1204-1207.
UV 420 color = UV 420/% solids

As seen, a wide variety of combinations of dextrose, citric acid and malto-oligosaccharide can be used to produce low-calorie oligosaccharides. The foregoing data also demonstrates how dextrose aids in extrusion, inasmuch as samples with little or no added dextrose are very dark and difficult to extrude. (It should be noted that each of the MALTRIN® products contains some dextrose). Samples with high levels of dextrose became hard glasses upon drying, thus making downstream processing more difficult. The best results were seen when MALTRIN® M100 and 25% or 12.5% added dextrose was used.

All of the samples incorporating the product of the invention had a higher average molecular weight and number average molecular weight than samples that were extruded only with glucose and citric acid.

### EXAMPLES 19-24

This example illustrates the effect of varying the level of citric acid catalyst in the preparation of dextrinized oligosaccharides.

A mixture of MALTRIN® M100/dextrose monohydrate/citric acid (the dry solid weight ratio of maltodextrin: dextrose being 4:1) was made by mixing 640 lbs of MALTRIN® M100 with 160 lbs of dextrose monohydrate and citric acid. The resulting blend was then automatically fed into a 57 mm twin screw Wenger TX-57 extruder at a rate of 111 lbs. per hour. Water was also fed to the extruder barrel at a rate of 12 lbs. per hour. The total moisture level of the feed was 18% (7% for the starting material, 11% from added to the extruder water). The extruder barrel temperature was monitored in five zones, according to the following table:

| | |
|---|---|
| Zone 1 | 57° C. |
| Zone 2 | 62° C. |
| Zone 3 | 59° C. |
| Zone 4 | 172° C. |
| Zone 5 (die head) | 172° C. |

The internal sample temperature at the die head was approximately 200 to 210° C. Low shear extruder screws were used. The extruder screw speed rate was 401 A single, 17 mm dye opening was used at the die head. The percent motor load for the extruded sample was 56%. A vacuum of 13 inches of water (0.57 atm) was used. The following table represents the ingredients and conditions employed.

In each case, the extruded product was a puffy, golden yellow solid material. The material was allowed to cool and ground to a golden yellow powder. The samples were analyzed yielding the following results. Color measurements are dyed on the international standard promulgated by the Commission Internationale d'Eclairage (CIE)

| | | | | | Max | Shaft | |
|---|---|---|---|---|---|---|---|
| | MD | | Dex- | Citric | Temp. | Speed | % Motor |
| Example | type | MD % | trose | Acid | (° C.) | (RPM) | Load |
| 19 | M100 | 80 | 20 | 0 | 199 | 401 | 56 |
| 20 | M100 | 80 | 20 | 0.075 | 209 | 401 | 60 |
| 21 | M100 | 80 | 20 | 0.125 | >179 | 401 | 56 |
| 22 | M100 | 80 | 20 | 0.25 | >182 | 401 | 58 |
| 23 | M100 | 80 | 20 | 0.5 | 204 | 145 | 48 |
| 24 | M100 | 80 | 20 | 1 | 208 | 144 | 58 |

| Example | DE | % Digest | Color (L) |
|---|---|---|---|
| 19 | 14.9 | 56.5 | 88 |
| 20 | 6.1 | 24.5 | 80 |
| 21 | 6 | 23.5 | 79 |
| 22 | 5.8 | 21.6 | 79 |
| 23 | 6.1 | 21.1 | 75 |
| 24 | 6.7 | 22.9 | 76 |

As seen, low levels of citric acid can be used to obtain the desired levels of digestibility. Citric acid aids in reducing digestibility and color formation.

### EXAMPLE 25

A sample of saccharide-derivatized oligosaccharides was prepared in accordance with Example 22. Five hundred grams of the product were slurried in warm water so that the total solids content was approximately 25%. Carbon SA-30 (Westvaco, Covington, Va.), 25 grams (5%) was added and the mixture was heated to 75° C. and held at this temperature for 4 hours. The solution was filtered through a celite bed to yield a yellow solution (Gardner Color=3, original color=9). The solution was then spray-dried on a Yamato lab spray drier to give 365 grams of an off-white product. The off-white powder had a Minolta L color value of 95 (compared with an initial value of 79). Chemical analysis of the product is shown in the table below:

| | Before carbon treatment | After carbon treatment |
|---|---|---|
| 3 hr % Digest # | 18.5 | 18.2 |
| 24 hr % Digest # | 21.6 | 20.6 |
| Dextrose | 1.50 | 1.46 |
| 2DE | 5.81 | 5.42 |
| % Citric Acid | 0.209 | 0.20 |
| % Levoglucosan | 1.57 | 1.49 |
| % 5-HMF* | 0.312 | 0.11 |
| % Ash | 0.17 | 0.39 |
| Color: | | |
| L | 79.4 | 95.1 |
| a | −1.4 | −6.1 |
| b | 25.5 | 12.0 |
| VISCOTEK: | | |
| Mn | 990 | 1,730 |
| Mw | 9,890 | 7,920 |

# Adapted from J. S. White et al.
*5-HMF = 5 hydroxymethyl furfural

As seen, carbon treatment removes color and 5-HMF, but otherwise does not essentially change the material. The flavor of the product is also greatly improved, as undesired off-flavors imparted by 5-HMF are essentially completely removed by the carbon treatment. The level of leveoglucosan, which can impart bitterness, also was reduced.

The decolored product, two polydextrose products, and a FIBERSOL product were obtained and evaluated. The results are shown on the following table:

| | | | | Reduced |
|---|---|---|---|---|
| | | | | Calorie |
| | Poly- | LITESSE III | | Oligo- |
| | dextrose | Polydextrose | FIBERSOL-2 | saccharide |
| Dextrose | 8.4 | 0.18 | 13.4 | 5.4 |
| Equivalent | | | | |
| % Free Glucose | 3.70 | 0 | 2.07 | 1.46 |
| % Levoglucosan | 1.26 | 1.42 | 0.20 | 1.49 |
| % 5-HMF | 0.68 | 0.35 | N.D.** | 0.11 |
| % Citric Acid | 0.66 | 0.002 | 0 | 0.1 |
| Color L Value | 93.80 | 96.36 | 94.71 | 95.05 |
| (White) | | | | |
| Color b Value | 13.68 | 6.11 | 12.38 | 12.00 |
| (Yellow) | | | | |
| Molecular Weight | 530 | 190 | 660 | 1,730 |
| (Mn) | | | | |
| Molecular Weight | 1,300 | 1,050 | 2,620 | 7,920 |
| (Mw) | | | | |
| Highest | 4 | 4 | 9 | 11 |
| Detectable | | | | |
| Oligosaccharide* | | | | |
| 24 Hour | 5.7 | 6.2 | 7.4 | 21.0 |
| Digestibility (%) | | | | |

*As detected by capillary electrophoresis
**Not determined

It is thus seen that the product of the invention is higher in molecular weight and comparable in color to polydextrose and FIBERSOL commercial products. Because of this relatively increased molecular weight, the product of the invention more closely resembles a maltodextrin. The product thus suitable for use in a wider range of applications.

### EXAMPLE 26

A sweetener is prepared by blending 965 grams of the spray-dried product of Example 25 with 35 grams calcium saccharin.

### EXAMPLE 27

A sweetener is prepared by blending 700 g of the spray-dried product of Example 25 with 300 g of sucralose.

### EXAMPLE 28

A pharmaceutical formulation is prepared by blending 10 grams acetaminophen with 100 grams of the spray-dried, carbon treated product prepared in accordance with Example 25. The resulting mixture is granulated and encapsulated.

### EXAMPLE 29

A 70/30/1 Limit Dextrin/Dextrose (anhydrous)/citric acid blend was made by mixing 700 g of limit dextrin with 300 g of anhydrous dextrose and 10 g of citric acid thoroughly in a Hobart mixture. The resulting blend was then manually fed into an 18 mm twin screw Leistritz extruder. The extruder barrel temperature was monitored in 6 zones according to the following table:

| | |
|---|---|
| Zone 1 | 50° C. |
| Zone 2 | 160° C. |
| Zone 3 | 180° C. |
| Zone 4 | 200° C. |
| Zone 5 | 200° C. |
| Zone 6 (die head) | 200° C. |

Low shear extruder screws were used. The extruder screw speed rate was 200 RPM. A single, 3 mm die opening was used at the die head. The motor load for the extruded sample was 50%. An off-white solid material was extruded. The material was allowed to cool, and ground to a off-white powder. The in vitro digestibility of the sample was 62% after 2.5 hours of enzyme treatment.

### EXAMPLE 30

Example 29 was repeated, except that the extruder screw speed was 100 RPM. The motor load was 75%. A light yellow solid material was extruded, was allowed to cool, and was ground to a light yellow powder. The in vitro digestibility of the sample was 67% after 2.5 hours of enzyme treatment.

### EXAMPLE 31

Example 30 was repeated, except that the motor load was 50%. An off white solid material was extruded, was allowed to cool, and was ground to an off white powder. The in vitro digestibility of the sample was 43% after 2.5 hours of enzyme treatment.

### EXAMPLE 32

Example 31 was repeated, except that the extruder screw speed was 200 RPM. The motor load remained at 50%. An off white solid material was extruded, was allowed to cool, and was ground to an off white powder. The in vitro digestibility of the sample was 43% after 2.5 hours of enzyme treatment

Thus, it is seen that the invention provides a product that is improved in many respects over known products such as polydextrose. The product of the invention finds applicability as a bulking agent and in numerous other uses.

While particular embodiments of the invention have been shown, it will be understood that the invention is not limited thereto since modifications may be made by those skilled in the art, particularly in light of the foregoing teachings. No unclaimed language should be regarded as limiting the scope of the invention. All references cited herein are hereby incorporated by reference in their entireties.

1-10. (canceled)

11. A process for preparing a mixture of saccharide-derivatized oligosaccharides, comprising: providing a saccharide product having an average degree of polymerization of 1 to 4; providing a mixture of malto-oligosaccharides in which at least a portion of the malto-oligosaccharides in said mixture have a degree of polymerization greater than 5; and derivatizing said mixture of malto-oligosaccharides with said saccharide product to form a mixture of saccharide-derivatized oligosaccharides by extruding a blend of said mixture of malto-oligosaccharides and said saccharide product under extrusion conditions sufficient to form a mixture of saccharide derviatized oligsaccharides.

12. A process according to claim 11, said derivatization being catalyzed with an acid.

13. A process according to claim 12, said acid being selected from the group consisting of citric acid, acetic acid, adipic acid, fumaric acid, gluconic acid, lactic acid, malic acid, phosphoric acid, and tartaric acid.

14. A process according to claim 14, said acid being citric acid.

15. A process according to claim 14, said acid being lactic acid.

16. A process according to claim 11, said blend being a spray-dried mixture of said saccharide product and said malto-oligosaccharide.

17. A process according to claim 11, including the step of selecting an amount of saccharide product insufficient to yield a liquid product from said extruder but sufficient to avoid charring said product.

18. A process according to claim 11, further comprising decolorizing said mixture of saccharide-derivatized oligosaccharides.

19. A process according to claim 18, further comprising the step of spray-drying said decolored mixture.

20. A spray-dried product formed in accordance with claim 19.

21. A process according to claim 11, said saccharide product and said malto-oligosaccharide mixture being provided in a single mixture.

22. A process according to claim 11, said saccharide product consisting essentially of dextrose.

23. A process according to claim 11, said saccharide product consisting essentially of a mixture of dextrose and a hydrogenated starch hydrolyzate, said product including an amount ranging from about 50% to about 95% by weight of said hydrogenated starch hydrolyzate.

24. A process according to claim 23, said hydrogenated starch hydrolyzate being sorbitol.

25. A process according to claim 22, said dextrose being in monohydrate form.

26. A process according to claim 11, said saccharide product consisting essentially of maltose.

27. A process according to claim 11, said saccharide product consisting essentially of maltotriose.

28. A process according to claim 11, said saccharide product consisting essentially of maltotetraose.

29. A process according to claim 11, said saccharide product comprising a mixture of dextrose and at least one other saccharide.

30. The product formed by the process of claim 11.

31. The product formed by the process of claim 23.

32. A process for preparing a saccharide derivatized oligosaccharide, comprising: providing an oligosaccharide having a degree of polymerization of at least 5; selecting an amount of saccharide product effective to derivatize said oligosaccharides via extrusion, said amount being sufficient to prevent significant charring of the derivatized product but insufficient to yield a liquid product upon extrusion; and extruding a mixture of said oligosaccharide and said saccharide to thereby derivatize said oligosaccharides.

33. A process for preparing a mixture of saccharide derivatized oligosaccharides, comprising: providing a mixture of malto-oligosaccharides in which at least a portion of the malto-oligosaccharides in said mixture have a degree of polymerization greater than 5; selecting an amount of a saccharide product effective to derivatize said mixture of malto-oligosaccharides via extrusion, said amount being sufficient to prevent significant charring of the derivatized product but insufficient to yield a liquid product upon extrusion; and extruding a mixture of said saccharide and said malto-oligosaccharides mixture to thereby derivatize said mixture of malto-oligosaccharides.
